(19) United States
(12) Patent Application Publication
(10) Pub. No.: US 2010/0249925 A1
(43) Pub. Date: Sep. 30, 2010

(54) ARTICLE AND A METHOD OF SURFACE TREATMENT OF AN ARTICLE

(75) Inventors: Gordon Blunn, North Herts (GB); Yaser Ghani, London (GB); Paul Unwin, Herts (GB)

Correspondence Address: HARNESS, DICKEY & PIERCE, P.L.C., P.O. BOX 828, BLOOMFIELD HILLS, MI, 48303, US

(73) Assignee: UCL BUSINESS PLC, London (GB)

(21) Appl. No.: 12/526,472

(22) PCT Filed: Feb. 8, 2008
(86) PCT No.: PCT/GB2008/000459

§ 371 Date: May 13, 2010

(30) Foreign Application Priority Data
Feb. 9, 2007 (GB) 0702577.8

(51) Int. Cl. A61F 2/02; B05D 3/14

(52) U.S. Cl. 623/11.11; 427/2.24

(57) ABSTRACT

A method of surface treatment of at least part of a surface of an implant, said method comprising: electrochemical deposition of a layer containing calcium and phosphorus ions onto a metallic substrate; and incorporation of a therapeutic agent into said electrochemically deposited layer and an implant so treated.

### BACKGROUND

The present invention relates to coatings on articles including prosthetic devices, in particular coatings on orthopaedic implants, with the incorporation into the coating of a therapeutic agent, particularly an antibacterial agent, preferably silver. The coating of articles with therapeutic agents has many uses including, but no limited to use on implants. Other uses may include surface treating items used in dentistry, for example. The invention is described below in relation to implants however.

Implants and in particular bone implants are being used more and more. The use of bone replacement implants for bone fractures or the use of supports for weakened bones is now commonplace. Furthermore, implants for the replacement of bone which has been removed due to a tumour (e.g. a bone (marrow) tumour) or for joint replacement is also becoming increasingly common. The use of biomimetic coatings on such implants is widespread and this helps in the incorporation of the implant into the bone and surrounding tissue.

Unfortunately rates of infection following implantation of prosthetic devices are still quite high. Infection rates run at about 2-4% for standard bone implants (such as hips) whereas more complicated and larger implants have an infection rate of about 10%. The larger rate for bone tumour implants is in part due to the likelihood of radiotherapy or, more frequently, chemotherapy taking place shortly after the implant has been implanted. These treatments suppress the immune system of the patient raising the chance of infection. The current rate of infection is as high as 30% when radiotherapy is used in bone tumour treatment.

It is known to apply a layer of hydroxyapatite (HA) onto implants using plasma spraying to act as a biomimetic layer. Hydroxyapatite is similar to naturally occurring apatite and a coating on an implant of hydroxyapatite (or other crystalline layer containing calcium and phosphorus) produces a surface of an implant which readily integrates with the surrounding bone and tissue after being implanted. It may only be necessary to coat part of a surface of an implant (which is usually metallic such as Ti6A14V).

In plasma spraying of hydroxyapatite a jet of ionised gas is formed into a plasma flame. Crystalline hydroxyapatite powder is fed into the plasma stream and melts. The molten particles are projected onto the outer surface of the implant and adhere to the surface of the implant. The use of plasma sprayed hydroxyapatite coatings has been approved as having the necessary physical properties for use on an implant.

A layer of sprayed hydroxyapatite would typically have a Ca:P ratio of about 1.67 and is quite dense.

### SUMMARY OF INVENTION

It is desirable to incorporate therapeutic agents in a surface coating of an implant.

The present invention provides a method of surface treatment of at least part of a surface of an article, said method comprising: electrochemical deposition of a layer containing calcium and phosphorus ions onto an electrically conductive substrate; and incorporation of a therapeutic agent into said electrochemically deposited layer.

Thus the therapeutic agent is incorporated into a relatively porous layer (the calcium and phosphorus ion containing layer) such that the therapeutic agent, in use, will leach out of that layer slowly over time. Furthermore, because the process can be carried out at room temperature, temperature induced harm to the therapeutic agent is unlikely. The electrochemically deposited layer replaces or augments traditional plasma sprayed hydroxyapatite.

Preferably the incorporation of a therapeutic agent occurs at least partly during the electrochemical deposition. In this way the number of steps in the manufacture of the implant are reduced. In this way the therapeutic agent may be incorporated into the crystalline lattice of the hydroxyapatite.

Additionally or alternatively the incorporation of a therapeutic agent occurs at least partly after the electrochemical deposition. This option allows a higher concentration of silver to be incorporated in the layer containing calcium and phosphorus ions.

Preferably, prior to the electrochemical deposition, a mineral is applied containing calcium and phosphorus, preferably hydroxyapatite, onto the metallic substrate by plasma spraying. Such a layer has good physical properties, in particular adhesion and strength and thereby provides a reliable surface on which to apply the electrochemically deposited layer containing calcium and phosphorus ions.

Preferably the layer containing calcium and phosphorus ions comprises hydroxyapatite and the electrochemical deposition comprises the deposition of brushite. The brushite can then converted into hydroxyapatite by soaking in a aqueous solution of sodium hydroxide.

Preferably the implant is an orthopaedic implant, preferably a bone tumour implant or a joint replacement implant. These type of implants are particularly suited to the present invention because of their inherent high cost and the risk of infection.

Preferably the therapeutic agent is silver. Silver is a potent antibacterial agent with a broad spectrum of activity and has been safely used in medicine for many years.

The article or implant resulting from the above method achieves many of the same advantages.

The present invention further provides an article comprising: on at least part of an electrically conductive substrate an electrochemically deposited layer containing calcium and phosphorus ions, wherein a therapeutic agent is incorporated within said electrochemically deposited layer.

The present invention further provides an article comprising: an outer coating of calcium and phosphorus containing crystals on an electrically conductive substrate; and a therapeutic agent incorporated within and/or between said crystals.

This implant advantageously allows leaching of the therapeutic agent out of the outer coating in a controlled manner over a large amount of time. Preferably the therapeutic agent is silver and is present in an amount of greater than 0.2 atomic percent of the outer layer. This provides reasonable antibacterial activity over a useful amount of time. More preferably the silver is present in an amount greater than 2%.

### BRIEF DESCRIPTION OF DRAWINGS

Embodiments of the invention will now be described, by way of example only, with reference to the accompanying schematic drawings in which:

FIG. 1 is a scanning electron micrograph of an electrochemically deposited layer of hydroxyapatite on a Ti6A14V substrate;

FIG. 2 is a scanning electron micrograph of a plasma spray deposited coating of hydroxyapatite on a Ti6A14V substrate;

FIG. 3 is a scanning electron micrograph of a coating according to example 1 of the present invention in which an electrochemically deposited layer of hydroxyapatite has been dipped in a solution of silver nitrate;

FIG. 4 illustrates a coating in accordance with example 3 of the present invention in which an outer layer is formed by concurrent electrochemical deposition of a calcium and phosphorus containing mineral with silver followed by soaking in silver nitrate; and

FIG. 5 is a graph showing the results of a bacterial inhibition test of examples of the present invention and comparative examples.

### EMBODIMENTS OF THE INVENTION

Due to its application at high temperatures and its low porosity, it has been found that the incorporation of therapeutic agents in plasma sprayed hydroxyapatite is problematic.

The present inventors have found that it is possible to incorporate therapeutic agents in electrochemically deposited minerals which contain calcium and phosphate. The thus deposited therapeutic agents are released in a controlled and sustained manner under physiological conditions.

It is thought that electrochemically deposited calcium phosphate minerals are more porous than similar plasma sprayed coatings and are able to absorb and entrap more of the therapeutic agents. Furthermore, the higher crystallinity of electrochemically deposited layers containing calcium and phosphate ions than the crystallinity of similar coatings prepared by plasma spraying enables the therapeutic agents to be trapped between crystals of the calcium and phosphate containing mineral. Therapeutic agents may also be trapped within the crystalline lattice of the coating material displacing other ions such as calcium or phosphate.

The present inventors have found that the therapeutic agents can be incorporated into the calcium phosphate mineral either at the time of its formation (i.e. by adding a substance to the solution used in the electrochemical deposition) or by soaking the coating in a solution after it has been electrochemically deposited either before or after (or both) conversion to another mineral.

The experimental results described below are carried out for an antibacterial agent, particularly silver. Other metallic ions such as copper and zinc may also have an antibacterial effect. However, therapeutic agents which can be incorporated in the above way include osteoconductive, osteoinductive and antimicrobial agents, but the method is particularly suited to the incorporation of metal ions, in particular antibacterial agents such as silver. Other agents include antibiotics and bone morphogenic proteins. One or more of such substances may be incorporated. Indeed, the therapeutic agents can be incorporated in both ways described below (i.e. during electrochemical deposition or after) in the same coating. Tests have shown that the therapeutic agents are active for longer in such coatings.

In the examples of the invention given below a layer of hydroxyapatite is formed on shotblasted discs of Ti6A14V which acts as a metal substrate. The hydroxyapatite is formed first by preparing a calcium phosphate solution which was used for electrochemical deposition. This resulted in a layer of brushite being formed on the Ti6A14V. This brushite was then converted to hydroxyapatite by placing the disc in 0.1M sodium hydroxide solution for 72 hours. However, the present invention is not limited to this specific methodology and coatings containing calcium and phosphorus can be used other than hydroxyapatite or the hydroxyapatite can be deposited directly on the metal substrate by electrochemical deposition. More amorphous coatings of hydroxyapatite will solubilise at a faster rate than crystalline coatings. The rate of release of the therapeutic agent from more amorphous coatings. The rate of release of the therapeutic agent from more amorphous coatings will therefore be faster increasing the concentration of the therapeutic agent locally. Other calcium phosphate coatings where this technology can be applied include alpha and beta tricalcium phosphate which again would solubilise faster than crystalline hydroxyapatite.

Furthermore, although solid Ti6A1V discs have been used as a metal substrate for the experiments, the invention is not limited to use of that shape or alloy. Different electrically conductive materials such as different alloys may also be suitable. Furthermore, the metal substrate can be provided as a coating on a polymeric body such as a polyethylene or polyurethane body. Additionally it is possible directly to coat certain polymers such as polyetheretherketone (PEEK) using this method. Because the electrochemical deposition process is a process which can be carried out at low temperatures, even at room temperature, this process is suitable for such bodies with low melting points.

Although the experiments were carried out with certain salt solutions, it should be understood that other salt solutions can also be used.

Compared to plasma applied hydroxyapatite, the electrochemically deposited hydroxyapatite has a higher Ca:P ratio. The ratio in electrochemically deposited hydroxyapatite is greater than 1.6, preferably greater than 1.7 and up to 2.1 (preferably between 1.7 and 2.0, more preferably between 1.7 and 1.8) whereas with plasma sprayed hydroxyapatite the ratio is generally around 1.67. Furthermore, electrochemically deposited hydroxyapatite is more porous that the plasma sprayed version and is less crystalline.

A further possibility, of which there is not an example below, is first to coat the implant with a hydroxyapatite coating using plasma spraying as is usual. This type of coating has good physical characteristics, particularly strength and adherence. Following that coating another outer layer of hydroxyapatite can be attached using the electrochemical deposition and incorporation of therapeutic agent as described below. Clearly in some instances it may not be necessary to coat an entire implant and only part of the implant could be coated.

The present invention is applicable to all types of prosthetics. These include all types implants and in particular orthopaedic implants including bone tumour implants or joint replacement implants.

### Comparative Example 1

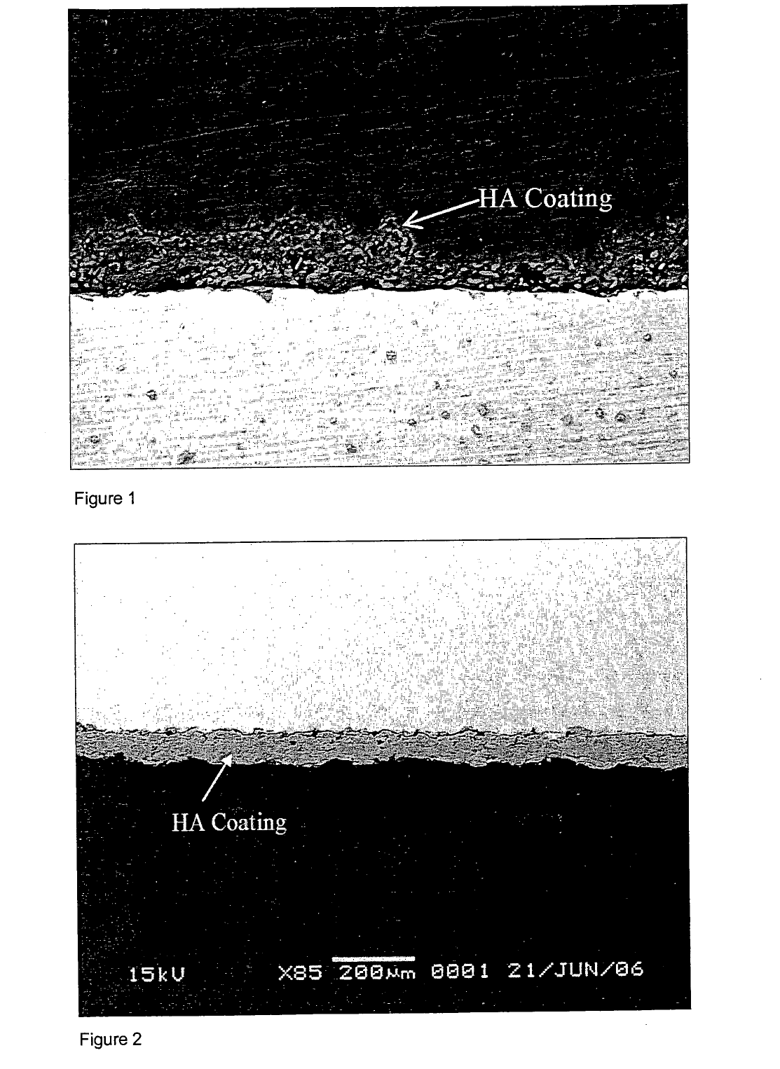

A calcium phosphate (CaP) solution was prepared for electrochemical deposition of hydroxyapatite onto a shotblasted 10 mm×3 mm Ti6A14V disc. A layer of brushite was then deposited on the discs by electrochemical deposition using that solution. The calcium phosphate solution was made by dissolving 30 grams of Ca(H₂PO₄)₂ in 1 litre of distilled water i.e. a 0.12M solution. The pH of the solution was pH 3.4. A platinum anode was used and the titanium disc attached to the cathodic terminal. Both the cathode and the anode were immersed in the solution and a current 200 mA/cm² was used for 10 minutes. This was carried out at room temperature. The brushite was then converted to hydroxyapatite by placing the disc in 0.1 M sodium hydroxide solution for 72 hours. FIG. 1 shows a scanning electron microscope (SEM) of the thereby produced layer.

The layer was 32.98 μm (+/−2.5 μm) thick and the Ca:P ratio was 1.71.

### Comparative Example 2

A layer of hydroxyapatite was sprayed deposited on a shotblasted 10 mm×5 mm Ti6A14V disc.

The thereby produced coating between 30-70 μm (+/−2.22 μm) thick and the Ca:P ratio of between 1.5-1.7.

### Comparative Example 3

A coating was prepared in the same way as the plasma sprayed coating of comparative example 2. The disc was then immersed in an AgNO₃ solution for 24 hours. The silver nitrate solution was made by adding 200 mg/200 ml i.e. a 0.0058M solution was used. This was done in room temperature in the dark.

FIG. 2 shows an SEM micrograph of the resulting structure. The amount of silver in the thus produced coating was measured at 0.10 atomic percent.

### Example 1

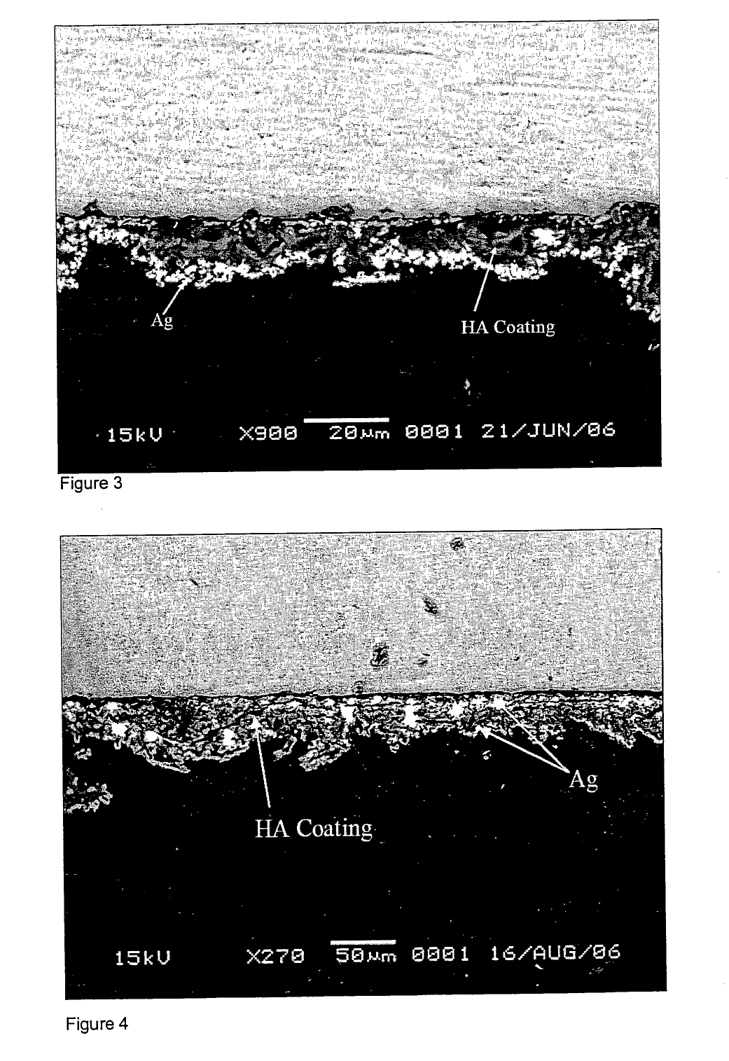

A disc was prepared in the same way as comparative example 1. This disc was then immersed in an AgNO₃ solution at a concentration of 200 mg/200 ml i.e. a 0.0058M solution for 24 hours at room temperature and in the dark. FIG. 3 shows an SEM micrograph of the resulting coating. As can be seen from the micrograph, a silver layer between the metal substrate and the hydroxyapatite coating can clearly be seen. The concentration of silver in the layer was measured as being 3.92 atomic percent.

### Example 2

A solution for electro-deposition of hydroxyapatite was prepared in accordance with comparative example 1. However, silver nitrate (AgNO₃) was added to the solution in an amount of 100 mg/200 mls of calcium phosphate solution prior to electrochemical deposition. Electrochemical deposition was then performed in the same way as in the comparative example 1 but in the dark. This produced a coating more rapidly and a thicker coating resulted. Silver was deposited within the crystal lattice of the HA. Using backscattered electron microscopy it was not possible to see any bright regions of silver deposition.

The resulting coating was measured as having a silver concentration of 0.38 atomic percent.

### Example 3

A layer of brushite was deposited as in comparative example 1. Silver was then applied by immersion in silver nitrate at 200 mg/200 mls in the dark. This was dried and then another layer of brushite was deposited and converted to HA in the same way as in comparative example 1 except this was carried out in the dark. Another layer of silver was applied by immersing in solvernitrate solution 200 mg/200 mls in the dark for 24 hours.

The resulting microstructure is illustrated in FIG. 4. As with FIG. 3, bright white silver layers can be seen in the micro graph and the silver was measured as being present at a level of 6.5 atomic percent in the coating of example 3.

### Experimental Results

As can be seen from the electromicro graphs and the results of energy dispersive x-ray and x-ray diffraction analyses it is possible to tell the difference between an electrochemically deposited layer of hydroxyapatite and a plasma spray coated layer. It is also clear from the results that soaking an electrochemically deposited layer of hydroxyapatite (with or without incorporated silver) results in a higher concentration of silver in the layer compared to the soaking of a plasma spray applied hydroxyapatite layer in the same solution.

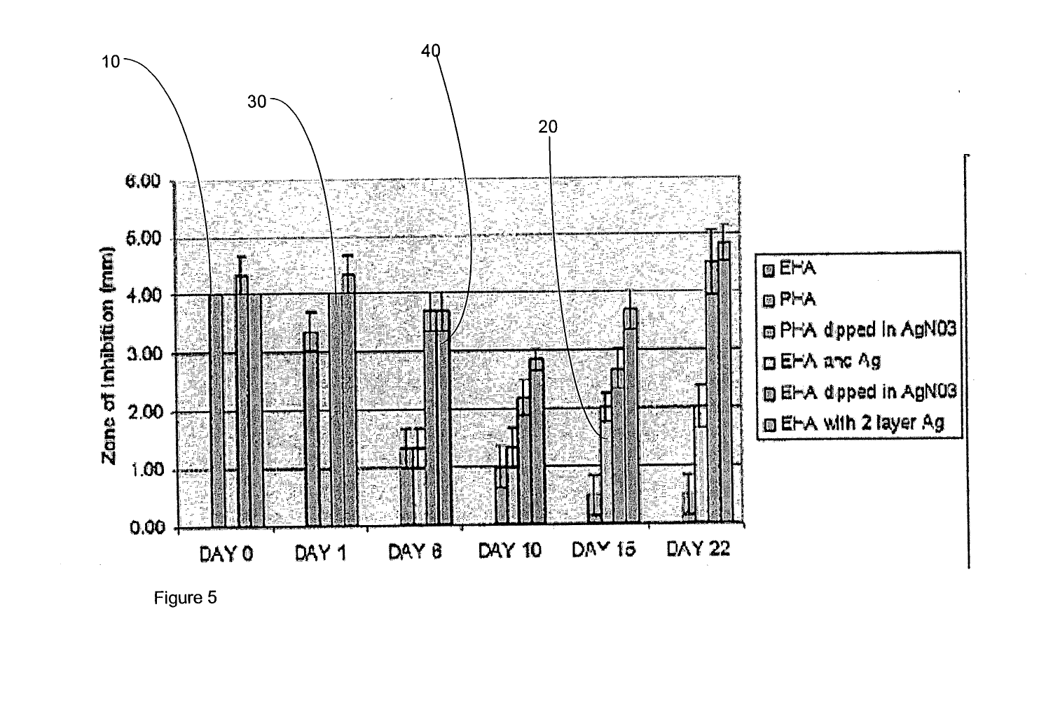

In order to test the efficiency of the various layers as an antibacterial agent discs of each of the examples were placed in 10 ml phosphate buffer solution, pH7.4 in a water bath at 37° C. to mimic physiological conditions. The phosphate buffer was changed daily and bacterial inhibition tests were carried out on these discs at days 0, 1, 6, 10, 15 and 22 using Staphyloccocus aureus (ATC 25923) with zone of inhibition measured from the edge of the disc to the edge of the clear zone. These results are illustrated in FIG. 5.

No zones of inhibition were seen in comparative examples 1 and 2. Comparative example 3 (labelled 10) shows a large zone of inhibition at day 1 but this decays rapidly to be barely present by day 22. It is thought that this is because the porosity of the plasma sprayed hydroxyapatite is not large enough to trap Ag ions.

In comparison, both examples 1 and 3 (labelled 30 and 40 respectively) showed high levels of anti bacterial activity throughout the 3 weeks of the test. Example 2 (labelled 20) showed no antibacterial activity at day 0 but this increased to a reasonable level by day 6 and continued to show a zone of inhibition greater than the comparative example 3.

For the examples where the electrochemically deposited hydroxyapatite was immersed in silver nitrate solution it is thought that the increased porosity and better crystallinity of the electrochemically deposited hydroxyapatite coatings results in their ability to absorb and entrap more Ag ions, releasing them in a controlled sustained manner over a period of the test. This shows that it is possible usefully to incorporate silver ions into the electrochemically deposited HA coating.

Because this technique can be carried out rapidly at room temperature it is possible to incorporate temperature sensitive therapeutic agents such as antibiotics and bone morphogenic proteins as well as the antibacterial agent described above. Furthermore, it is possible to use the technique on implants which may be temperature sensitive, such as those made of polymers with only a thin coating of metal to act as the substrate.

1.-42. (canceled)

43. A method of surface treatment of at least part of a surface of an article, said method comprising:
electrochemical deposition of a layer containing calcium and phosphorus ions onto an electrically conductive substrate; and
incorporation of a therapeutic agent into said electrochemically deposited layer.

44. The method of claim 43, wherein said incorporation of a therapeutic agent occurs at least partly during said electrochemical deposition, and a solution used for said electrochemical deposition comprises a substance for incorporation of said therapeutic agent.

45. The method of claim 44, wherein said substance comprises at least one of the following: silver ions, an antibiotic, bone morphogenic proteins.

46. The method of claim 44, wherein said substance comprises a silver salt, preferably silver nitrate or silver lactate.

47. The method of claim 43, further comprising, prior to said electrochemical deposition, applying a mineral containing calcium and phosphorus, preferably hydroxyapatite, onto said metallic substrate by plasma spraying.

48. The method of claim 43, wherein said electrochemical deposition comprises the deposition of brushite or hydroxyapatite.

49. The method of claim 48, wherein, if said electrochemical deposition comprises the deposition of brushite, converting said brushite to hydroxyapatite by soaking in an aqueous solution of sodium hydroxide.

50. The method of claim 49, wherein, following said incorporation of a therapeutic agent after deposition of brushite, a further layer of brushite is electrochemically deposited before said converting.

51. The method of claim 49, wherein said soaking comprises soaking for more than 10 hours.

52. The method of claim 43, wherein the substrate is a metal coating on a non-metal object.

53. An article comprising: on at least part of an electrically conductive substrate an electrochemically deposited layer containing calcium and phosphorus ions, wherein a therapeutic agent is incorporated within said electrochemically deposited layer.

54. The article of claim 53, further comprising, beneath said outer layer a layer of a mineral containing calcium and phosphorus, preferably hydroxyapatite, which was applied by plasma spraying.

55. An article comprising:
an outer coating of calcium and phosphorus containing crystals on an electrically conductive substrate; and
a therapeutic agent incorporated within and/or between said crystals.

56. The article of claim 55, wherein said outer coating has a Ca:P ratio of between 1.68 and 2.1.

57. The article of claim 55, wherein said therapeutic agent is silver and is present in an amount of greater than 0.2 atomic percent of said outer layer.

58. The article of claim 55, further comprising an inner coating of a calcium and phosphorus containing mineral, preferably hydroxyapatite, between said outer coating and said metallic substrate.

59. The article of claim 58, wherein the Ca:P ratio in said outer layer is greater than in said inner layer.

60. The article of claim 58, wherein the porosity of said outer layer is greater than of said inner layer.

61. The article of claim 55, wherein the crystallinity of said outer layer is greater than that of said inner layer.

62. The article of claim 55, wherein the substrate is a metal coating on an non-metal object.
